(19) United States
(12) Patent Application Publication
(10) Pub. No.: US 2006/0224212 A1
(43) Pub. Date: Oct. 5, 2006

(54) Neural electrode array

(76) Inventor: Philip R. Kennedy, Duluth, GA (US)

Correspondence Address: BRYAN W. BOCKHOP, ESQ., 2375 MOSSY BRANCH DR., SNELLVILLE, GA, 30078, US

(21) Appl. No.: 11/096,897

(22) Filed: Apr. 1, 2005

(51) Int. Cl. A61N 1/18

(52) U.S. Cl. 607/54

(57) ABSTRACT

A neural electrode array includes an electrode support member, a conductor and at least one anchor structure. The electrode support member is substantially rigid and non-conductive and defines a plurality of spaced-apart holes passing therethrough. An electrically conductive contact is disposed adjacently to each hole. The conductor uniquely connects each contact to a bus. The anchor structure includes a portion for engagement with tissue that is capable of maintaining the support member in a substantially fixed relationship with a neural region.

### BACKGROUND

1. Field of the Invention

The present application relates to retinal prostheses and, more particularly, to an electrode for retinal stimulation.

2. Description of the Related Art

The human eye has a number of components. These include the cornea, iris, pupil, lens, optic nerve, vitreous humor, the sclera and the retina. The cornea is the clear front window of the eye that transmits and focuses light into the eye. The iris is the colored part of the eye that helps regulate the amount of light that enters the eye. The pupil is the dark aperture in the iris that determines how much light is let into the eye. The lens is the transparent structure inside the eye that focuses light rays onto the retina. The vitreous humor is a clear, jelly-like substance that fills the middle of the eye. The retina is the nerve layer that lines the back of the eye, senses light, and creates impulses that travel through the optic nerve to the brain. There is a small area, called the macula, in the retina that contains special light-sensitive cells. The macula allows us to see fine details clearly. The sclera is commonly known as “the white of the eye.” It is the tough, opaque tissue that serves as the eye's protective outer coat. The retina includes several layers of cells. At the light-sensing surface of the retina is a layer of photo-receptor cells referred to as “rods” and “cones.” Beneath the photo-receptor cells several layers of intermediary cells (such as pedicules spherules, horizontal bipolar cells and amacrine cells) that transmit light-induced events from the photo-receptor cells to a layer of ganglion cells. The ganglion cell axons form the optic nerve, which travels from the eye and terminates in various regions of the brain, where the combined input is processed along multiple routes and ultimately results in the experience of sight. Essentially, the axons transmit light-induced events from the retina to the visual cortex in the brain.

Certain patients have healthy ganglion cells, but have degenerated photo-receptor cells. If the photo-receptor cells are substantially degenerated, then blindness results. If the patient's ganglion cells are healthy and intact, then artificial stimulation of the ganglion cells results in impulses being transmitted to the visual cortex, thereby generating perception of light.

Several intraocular retinal prosthetic devices have been proposed to combat the effects of certain types of progressive blindness. Such prostheses are intended to stimulate retinal ganglion cells whose associated photoreceptor cells have fallen victim to degradation by diseases such as macular degeneration or retinitis pigmentosa, two currently incurable but widespread conditions.

Retinal prostheses attempt to bypass degenerated photoreceptors by providing electrical stimulation directly to the underlying ganglion cells. Electrical stimulation of the ganglion cells by a retinal prosthesis attempts to mimic the electrical activity within a retinal ganglion cell corresponding to a visual stimulus of a photo-receptor cell. Direct stimulation of the ganglion cells may restore a measure of sight to patients with substantial photo-receptor cell degeneration.

A retinal prosthesis includes a source of electrical impulses that correspond to light that would be received by the eye. The impulses could be computer generated, using input from a camera to transmit corresponding impulses to an array of electrodes that interface with the ganglion cells in the patient's eye.

Several references disclose systems for the electrical stimulation of the retina by a retinal electrode array held against the retina, including systems for capturing a video image, transferring the image wirelessly into a living body and applying the image to a retinal electrode array. One proposed electrode array includes wire-type electrodes that stick into the layer of ganglion cells. One term for this type of an electrode array is a “pin cushion array.” The wire-type electrodes are held by a non-conductive frame that is implanted in the eye and are electrically connected to a ribbon cable that passes information from the computer to the electrode array. The electrodes themselves must be anchored to the retina with sufficient strength to accommodate physical agitation due to daily activity. One difficulty with such an array is that the anchoring may be insufficient, thereby allowing the electrodes to dislodge from the ganglion cells.

Therefore, there is a need for a retinal electrode array that is stable when applied to the retinal area of an eye.

### SUMMARY OF THE INVENTION

The present invention, in one aspect, is a neural electrode array that includes an electrode support member, a conductor and at least one anchor structure. The electrode support member is substantially rigid and non-conductive and defines a plurality of spaced-apart holes passing therethrough. An electrically conductive contact is disposed adjacently to each hole. The conductor uniquely connects each contact to a bus. The anchor structure includes a portion for engagement with tissue that is capable of maintaining the support member in a substantially fixed relationship with a neural region.

In another aspect, the invention is a retinal electrode array that includes a substantially rigid and non-conductive electrode support member defining a plurality of spaced-apart holes passing therethrough and an electrically conductive contact disposed adjacently to each hole. A conductor uniquely connects each contact to a bus. At least one sclera anchor structure, including a portion for engagement with sclera tissue, is capable of maintaining the support member in a substantially fixed relationship with a retinal area of an eye.

In another aspect, the invention is a device for transmitting electrical impulses to an optic nerve. A retinal electrode array is configured to receive growth of optic nerve cells into a plurality of electrodes. A bus includes a plurality of conductors, with each conductor in electrical communication with a different electrode of the plurality of electrodes. The bus is capable of transmitting electrical pulses from an electrical pulse source to each of the plurality of electrodes.

In another aspect, the invention is a neural electrode that includes a substrate that defines a hole passing therethrough into which nerve tissue may grow. An electrode contact is exposed to the hole. An electrical conductor electrically couples the electrode contact to a source of electrical stimulation.

In yet another aspect, the invention is a method of transmitting electrical pulses to optic nerve cells, in which a retinal electrode array is applied to a retinal area of an eye. The retinal electrode array includes a rigid and nonconductive support member that defines a plurality of holes passing therethrough. The retinal electrode array also includes a plurality of electrodes, each in contact with a separate one of the plurality of holes, and a plurality of conductors, each conductor capable of placing a different line of a bus in electrical contact with a separate one of the plurality of electrodes. Nerve tissue is allowed to grow into at least a portion of the plurality of holes, thereby establishing contact between nerve cells and the plurality of electrodes. A stimulus is applied to at least one of the electrodes, thereby stimulating a nerve cell.

These and other aspects of the invention will become apparent from the following description of the preferred embodiments taken in conjunction with the following drawings. As would be clear to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram of an electrode matrix implanted in an eye.

FIG. 2 is a cross sectional diagram of an electrode matrix in which ganglion cells have grown into holes in the array.

FIG. 3A is a top plan view of a portion of an electrode matrix connected to a ribbon cable.

FIG. 3B is a top plan view of a portion of an alternative electrode matrix connected to a receiver.

FIG. 4A is a plan view of an electrode matrix.

FIG. 4B is a cross-sectional view of the electrode matrix shown in FIG. 4A, taken along line 4B-4B.

FIG. 5A is a cross-sectional view of one electrode arrangement.

FIG. 5B is a cross-sectional view of a second electrode arrangement.

FIG. 6A is a top plan view of a mono-polar electrode arrangement.

FIG. 6B is a top plan view of a bipolar electrode arrangement.

FIG. 7 is a schematic diagram of one embodiment of the invention being applied to a visual cortex.

### DETAILED DESCRIPTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

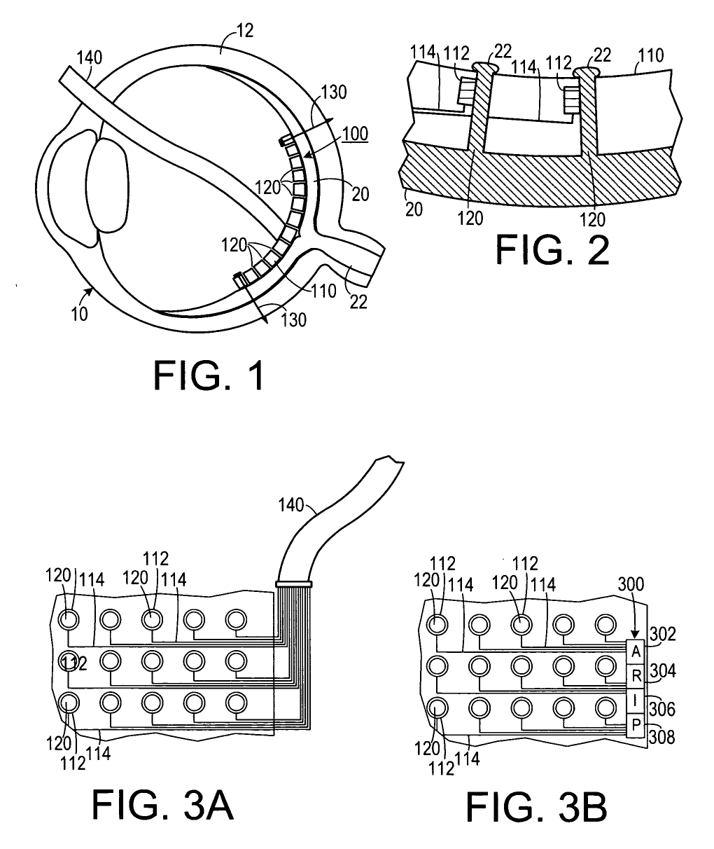

As shown in FIG. 1, one exemplary embodiment of the invention is a retinal electrode array 100 that is placed in the ocular system 10 of a user. The ocular system 10 includes a sclera 12, a retina 20 and an optic nerve 22. The retinal electrode array 100 includes an electrode support member 110 that is made of a substantially rigid non-conductive material that is non-reactive with surrounding eye tissues. The electrode support member 110 defines a plurality of holes 120 passing therethrough. The holes 120 are spaced apart in an ordered manner. The electrode support member 110 may be held in place against at least a portion of the retina 20 with at least one anchor pin 130. In one illustrative embodiment, the holes 120 are about 10 microns in diameter and are spaced apart in a range of about 10 to 200 microns.

The electrode support member 110 should be rigid and non-conductive. It could be made of materials such as: silica, silicon, amorphous glass, gallium arsenide and certain polymers (such as liquid crystal polymers).

As shown in FIG. 2, a plurality of electrodes 112 are disposed adjacent to each of the holes 120. Ganglion cells 22 grow into the holes 120 and achieve contact with the electrodes 112. As the ganglion cells grow through the holes 120 they further stabilize the retinal electrode array 100 relative to the retina 20. To encourage the ganglion cells 22 to grow into the holes 120, a nerve growth factor, such as brain-derived neurotropic factor (BDNF) or ciliary neurotropic factor (CNTF), may be applied to the electrode support member 110 in the region of substantially each of the holes 120. A basement membrane matrix, such as MATRIGEL™, available from Becton, Dickenson and Company, 1 Becton Drive, Franklin Lakes, N.J. 07417, may be applied prior to applying the nerve growth factor. The basement membrane matrix will adhere to the electrode support member 110 and adsorb the nerve growth factor, thereby stabilizing it in the region of the holes 120.

As shown in FIG. 3A, each of the electrodes 112 is electrically coupled to a different electrical lead 114. All of the leads 114 form a data bus 140 that exits the eye 10. Impulses from a computer interface can then be applied to the electrodes 112 via the data bus 140. As shown in FIG. 3B, data can be transmitted to the electrodes 112 via a radio frequency receiver unit 300. The radio frequency receiver unit 300 could include an antenna 302, a receiver 304, processor 308 and an induction-coil driven power source 306.

The retinal electrode array 100 is applied to a retinal area of an eye using established retinal surgical techniques. Once the retinal electrode array 100 has been implanted, nerve tissue is allowed to grow into the plurality of holes 120. This establishes contact between nerve cells and the plurality of electrodes 112 and secures the retinal electrode array 100 to the retinal tissue. Once nerve tissue has grown into the holes 120, stimuli are applied to the electrodes 112 via the bus 140, thereby stimulating nerve cells and causing a sensation of light.

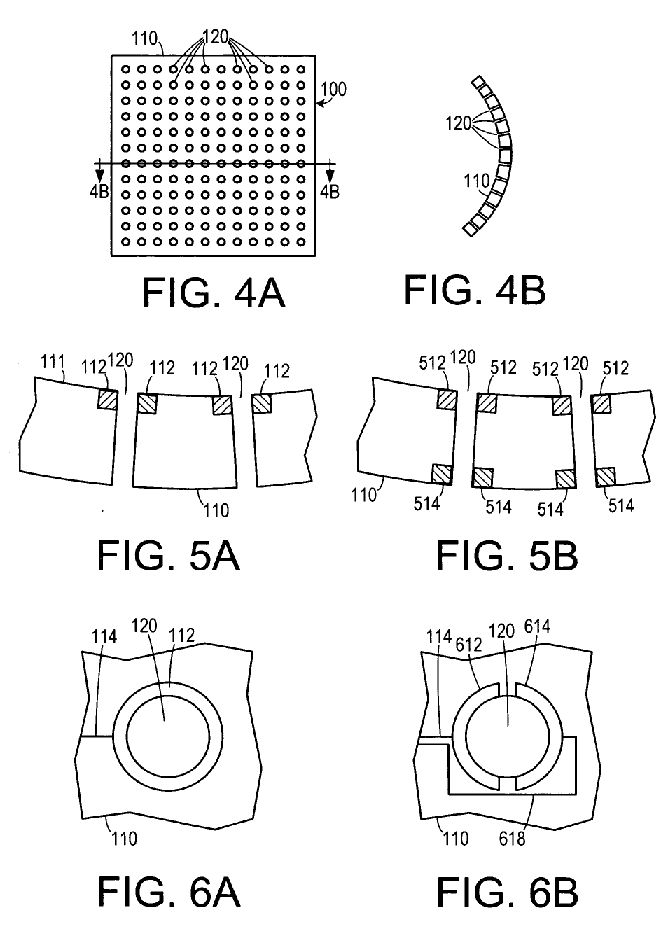

As shown in FIGS. 4A and 4B, one arrangement for an electrode array 100 has the holes 120 space apart evenly. They could be distributed in other patterns, such as circular, or even concentrated in predetermined areas of the electrode support member 110.

As shown in FIG. 5A, the electrodes 112 may be disposed along a top surface 111 of the electrode support member 110. In this arrangement, a ground electrode (not shown) would also be in electrical contact with the patient. Alternately, as shown in FIG. 5B, each hole 120 could include an electrode 512 and a spaced-apart ground 514, so that electrical impulses would be transmitted primarily along a path between the electrode 512 and the ground 514. A shown in FIG. 6A, the electrode 112 could encircle the hole 120. Alternately, as shown in FIG. 6B, the electrode 612 could be disposed around only a portion of the hole 120, with the ground 614 being disposed along an opposite portion of the hole 120. In this arrangement, the signal would stimulate nerve tissue primarily in the region between the electrode 612 and the ground 614 (which would be connected to a ground wire 618). The arrangements shown in FIGS. 5B and 6B may reduce the amount of cross-talk between electrodes.

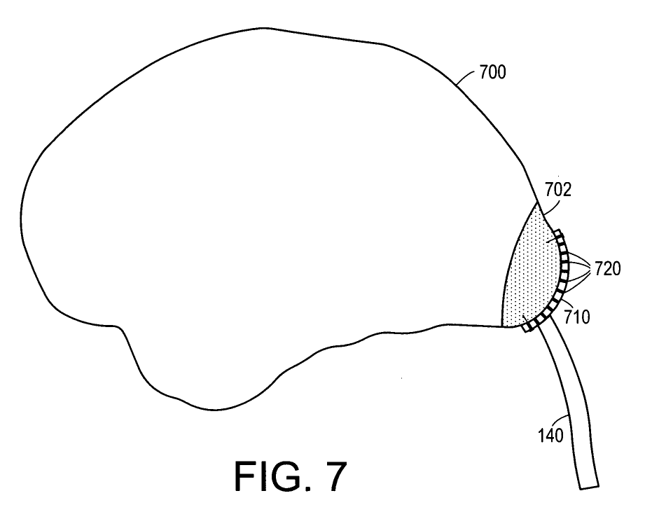

The invention is not limited to application to the retinal area. The invention can be applied to any neural region that processes multiple spaced-apart stimuli. In one example, as shown in FIG. 7, one embodiment of a neural electrode array 710, including a plurality of spaced-apart holes 720, is applied to the visual cortex 702 of a brain using established neurosurgical techniques. As is evident to those of skill in the neurological arts, the neural electrode may be used in many neural-computer interface applications, including those involving sensing neural impulses and stimulating motor neurons.

While the invention has been particularly shown and described with reference to a embodiment shown herein, it will be understood by those skilled in the art that various changes in form and detail maybe made without departing from the spirit and scope of the present invention as set for in the following claims. Furthermore, although elements of the invention may be described or claimed in the singular, the plural is contemplated unless limitation to the singular is explicitly stated.

What is claimed is:

1. A neural electrode array, comprising:
a. a substantially rigid and non-conductive electrode support member defining a plurality of spaced-apart holes passing therethrough;
b. an electrically conductive contact disposed adjacently to each hole;
c. a conductor that uniquely connects each contact to a bus; and
d. at least one anchor structure including a portion for engagement with tissue that is capable of maintaining the support member in a substantially fixed relationship with a neural region.

2. The neural electrode array, of claim 1, further comprising a nerve growth factor applied to substantially each of the holes.

3. The neural electrode array, of claim 2, wherein the nerve growth factor comprises brain-derived neurotropic factor.

4. The neural electrode array, of claim 2, wherein the nerve growth factor comprises ciliary neurotropic factor.

5. The neural electrode array, of claim 1, wherein the electrode support member comprises silicon.

6. The neural electrode array, of claim 1, wherein the electrode support member comprises a liquid crystal polymer.

7. The neural electrode array, of claim 1, wherein the electrode support member comprises an amorphous glass.

8. The neural electrode array, of claim 1, wherein the electrode support member comprises gallium arsenide.

9. The neural electrode array, of claim 1, wherein the electrode support member has a thickness in the range of from one-half millimeter to one millimeter.

10. The neural electrode array, of claim 1, where each of the holes has a diameter of about 10 microns.

11. The neural electrode array, of claim 1, wherein the neural region comprises a retinal region.

12. The neural electrode array, of claim 1, wherein the neural region comprises a neural cortex region.

13. The neural electrode array, of claim 12, wherein the neural cortex region comprises a visual cortex region.

14. A retinal electrode array, comprising:
a. a substantially rigid and non-conductive electrode support member defining a plurality of spaced-apart holes passing therethrough;
b. an electrically conductive contact disposed adjacently to each hole;
c. a conductor that uniquely connects each contact to a bus; and
d. at least one sclera anchor structure including a portion for engagement with sclera tissue that is capable of maintaining the support member in a substantially fixed relationship with a retinal area of an eye.

15. The retinal electrode array of claim 14, further comprising a nerve growth factor applied to substantially each of the holes.

16. The retinal electrode array of claim 15, wherein the nerve growth factor comprises brain-derived neurotropic factor

17. The retinal electrode array of claim 15, wherein the nerve growth factor comprises ciliary neurotropic factor.

18. The retinal electrode array of claim 14, wherein the electrode support member comprises silicon.

19. The retinal electrode array of claim 14, wherein the electrode support member comprises a liquid crystal polymer.

20. The retinal electrode array of claim 14, wherein the electrode support member comprises an amorphous glass.

21. The retinal electrode array of claim 14, wherein the electrode support member comprises gallium arsenide.

22. The retinal electrode array of claim 14, wherein the electrode support member has a thickness in the range of from one-half millimeter to one millimeter.

23. The retinal electrode array of claim 14, where each of the holes has a diameter of about 10 microns.

24. A device for transmitting electrical impulses to an optic nerve, comprising:
a. a retinal electrode array configured to receive growth of optic nerve cells into a plurality of electrodes; and
b. a bus that includes a plurality of conductors, each conductor in electrical communication with a different electrode of the plurality of electrodes, the bus capable of transmitting electrical pulses from an electrical pulse source to each of the plurality of electrodes.

25. The device of claim 24, further comprising at least one eye anchor structure including a portion for engagement with eye tissue that is capable of maintaining the retinal electrode array in a substantially fixed relationship with a retinal area of an eye.

26. A neural electrode, comprising:
a. a substrate that defines a hole passing therethrough into which nerve tissue may grow;
b. an electrode contact exposed to the hole; and
c. an electrical conductor that electrically couples the electrode contact to a source of electrical stimulation.

27. The retinal electrode of claim 26, further comprising a nerve growth factor applied to the hole.

28. The retinal electrode of claim 27, wherein the nerve growth factor comprises brain-derived neurotropic factor.

29. The retinal electrode of claim 27, wherein the nerve growth factor comprises ciliary neurotropic factor.

30. A method of transmitting electrical pulses to optic nerve cells, comprising the steps of:
a. applying a retinal electrode array to a retinal area of an eye, the retinal electrode array including a rigid and nonconductive support member defining a plurality of holes passing therethrough, a plurality of electrodes, each in contact with a separate one of the plurality of holes, and a plurality of conductors, each conductor capable of placing a different line of a bus in electrical contact with a separate one of the plurality of electrodes;
b. allowing nerve tissue to grow into at least a portion of the plurality of holes, thereby establishing contact between nerve cells and the plurality of electrodes; and
c. applying a stimulus to at least one of the electrodes, thereby stimulating a nerve cell.

31. The method of claim 30, further comprising the step of anchoring the retinal electrode array to sclera tissue of the eye.

32. The method of claim 30, further comprising the step of applying nerve growth factor to each of the holes to facilitate nerve growth into the holes.

33. The method of claim 32, wherein the applying step comprises the step of applying a basement membrane matrix to the holes.
